(19) United States
(12) Patent Application Publication
(10) Pub. No.: US 2024/0226442 A9
(43) Pub. Date: Jul. 11, 2024

(54) METHODS AND SYSTEMS FOR SAFE INJECTION OF DERMAL FILLER PROCEDURES

(71) Applicant: THE GENERAL HOSPITAL CORPORATION, Boston, MA (US)

(72) Inventors: Molly Wanner, Boston, MA (US); William Farinelli, Danvers, MA (US); Richard Rox Anderson, Boston, MA (US); Joseph A. Gardecki, Acton, MA (US); Matthew J. Beatty, Malden, MA (US); Guillermo J. Tearney, Cambridge, MA (US)

(21) Appl. No.: 18/547,917

(22) PCT Filed: Feb. 25, 2022
(86) PCT No.: PCT/US2022/017978

§ 371 Date: Aug. 25, 2023

Related U.S. Application Data
(60) Provisional application No. 63/153,510, filed on Feb. 25, 2021.

(51) Int. Cl. A61M 5/31; A61M 5/32; A61M 5/34

(52) U.S. Cl. CPC A61M 5/3134; A61M 5/329; A61M 5/3293; A61M 5/34; A61M 2205/50; A61M 2205/587

(57) ABSTRACT

An injection apparatus, including: a needle configured to be inserted into a tissue; a light source to deliver light to the tissue to generate reflected light; a detector to detect the reflected light from the tissue; a processor coupled to the detector and configured to: analyze the reflected light from the tissue to identify a tissue type associated with the reflected light, and provide an output to a user based on the identified tissue type.

### CROSS-REFERENCE TO RELATED APPLICATIONS

The present application is based on and claims priority from U.S. Patent Application Ser. No. 63/153,510, filed on Feb. 25, 2021, the entire disclosure of which is incorporated herein by reference.

### STATEMENT REGARDING FEDERALLY SPONSORED RESEARCH

N/A.

### BACKGROUND

Cosmetic soft-tissue fillers are substances injected into the face and elsewhere in the body to replace volume loss that occurs with aging. A potential complication of this procedure is the inadvertent injection of the filler into a blood vessel, which can have severe and unwanted effects. Currently, a clinical practice to avoid complications is to place the needle in the skin, pull back on the plunger and watch to see if blood enters the syringe and, if blood is not seen, the filler is then delivered into the skin. However, this procedure is time-consuming and may not always be reliable. Given that there are more than 3.5 million filler procedures completed each year, there is a need to make these procedures as safe and reliable as possible.

### SUMMARY OF THE INVENTION

Accordingly, the methods and systems disclosed herein address one or more issues identified above by providing an injection device and system, which detects the type of tissue at or near the tip of the injection needle and provides a warning to trained medical staff performing the injection if the needle tip is in or near a particular tissue type (e.g. blood or blood vessel).

In general, the needle-tip injection system uses light to detect vasculature penetration during the placement of a hypodermic needle and prior to injection. The system makes use of the differences in optical absorption between different tissue types (e.g. blood and surrounding tissues) to provide feedback for guiding the hypodermic needle to a safe placement location. It is established that light in certain regions of the visible spectrum is highly absorbed by specific targets, especially hemoglobin. Thus, as the concentration of hemoglobin increases, so too does the absorption of light at these particular wavelengths. Whole blood has the highest concentration of hemoglobin of any tissue, making it the highest absorber of light at certain wavelengths. It is also established that light may be scattered by tissues, and that this scattered light can be collected and analyzed. As the needle enters the tissue, light will be transmitted into the skin (e.g. by an external source or by a fiber at the needle tip that is coupled to a light source). Backscattered light reflected by the tissue will be collected by the device and sent to a detector. When the needle enters tissue that has low whole blood content, backscattering will be high, and the detector will collect a strong reflection signal. As the needle approaches a vessel, the signal will drop due to increased absorption of light from the blood hemoglobin. If the needle enters or touches a blood vessel, the light will be mostly absorbed by the whole blood. In this case, the amount of backscattered light sent to the detector will be low, which will result in a sudden loss of signal. This decrease in signal will be detected and analyzed, and a warning will be sent to an operator of the syringe, for example using a visual or audible signal.

The system includes, among other components, a light source and a detector. A 577 nm wavelength LED was chosen for certain embodiments because this wavelength allows optimal differentiation between blood hemoglobin and muscle myoglobin, although other wavelengths and sources such as laser diodes can be used instead of, or in addition to, the 577 nm LED light, for example a source which emits light with a wavelength in a range of 568-577 nm. The source is housed in the needle hub where it is connected to a fiber optic probe. Also housed in the needle hub is an optical detector and supporting electronics that capture and process the backscattered light. The detector is attached to a fiber optic probe that is similar to the fiber attached to the light source.

In the one embodiment, the needle probe includes two optical fibers, one to deliver the light from the source to the tissue and a second to collect the scattered light from the tissue and transmit it to a detector (see FIGS. 1-4). the fibers may be placed next to one other to optimize signal quality. The fiber-containing needle probe may be fitted to a hypodermic needle, cannula, or other tubular element, where the ends of the fibers are located at or near the tip of the needle. During placement of the needle, light from the delivery fiber illuminates the tissue and the second (collection) fiber collects the backscattered light, which is transmitted to an optical detector. The use of two fibers in this embodiment provides optical isolation and allows detection of small changes in the back-reflected light as well as the ability to differentiate between different tissue types. A lens can be connected to one or both ends of the collection fiber to enhance light collection efficiency. In another embodiment, a single fiber can both deliver the illumination beam and collect the scattered light. In other embodiments of the device, a wireless transmitter may be included such that the disposable needle hub may house only the LED, photodiode, amplifier, optional filter electronics, and a low power transmitter; embodiments such as this reduce costs associated with the disposable element and allows for further device miniaturization.

In various embodiments, the optical detector may be coupled (e.g. in a wired or wireless manner) to processing electronics that will analyze the data and provide a warning to the physician (e.g. audibly and/or visually) when the needle tip is in or near a blood vessel. Thus, when the needle, cannula, or other tubular element is inserted into a blood vessel, the physician will be alerted with an audible beep, a light flash, and/or other signal. Furthermore, the needle may be capable of integrating an early, “yellow light” warning system that alerts the injector that the needle is approaching a vessel and a “red light” warning when the needle is in the vessel. The needle can also be connected by Bluetooth or similar low power transmission protocol to a smart phone or similar receiver unit to deliver information about the tissue at or near the tip of the needle.

The device may include one or more of the following features:
The device uses optical data to differentiate between tissue types located in or under the skin and whole blood contained in a vessel, which may be based on a single optical measurement.
The device can alert the physician to the presence of blood in real-time guiding placement.
The device can be outfitted to accommodate different gauges of cannula and needles.
The device may house all of the necessary components in the needle hub. This allows the needle to be easily removed and changed for a different size needle or cannula, which adds important flexibility for the injector. In addition, this needle hub does not change the ergonomics of the needle injections. Furthermore, all components of the device are inexpensive and disposable.
The device utilizes a wavelength that has been optimized to differentiate between myoglobin and hemoglobin.
The devices utilize fibers that have been optimized for the ideal needle gauge for filler injections. Filler injections typically use 23-27 gauge cannulas and needles. In fact, lower gauge (23-25) cannulas are considered safer than 27 gauge cannulas. The fibers may be located in a longitudinal slot or groove on the outside of the needle in one embodiment or, in another embodiment, may be disposed within longitudinal channels within the wall of the needle shaft that are created by during needle formation. Based on initial experiments, the fibers in certain embodiments are placed next to each other to allow detection of the greatest amount of light.

In one embodiment, the disclosure provides an injection apparatus, including: a needle configured to be inserted into a tissue; a light source to deliver light to the tissue to generate reflected light; a detector to detect the reflected light from the tissue; a processor coupled to the detector and configured to: analyze the reflected light from the tissue to identify a tissue type associated with the reflected light, and provide an output to a user based on the identified tissue type.

In another embodiment, the disclosure provides a hub for use with an injection apparatus, including: a needle configured to be inserted into a tissue; at least one waveguide associated with the needle; and a connector for detachably connecting the hub to a flange.

In still another embodiment, the disclosure provides a method for injection, including: providing an injection apparatus including: a needle configured to be inserted into a tissue, a light source to deliver light to the tissue to generate reflected light, and a detector to detect the reflected light from the tissue; analyzing, using a processor coupled to the detector, the reflected light from the tissue to identify a tissue type associated with the reflected light; and providing, using the processor, an output to a user based on the identified tissue type.

In yet another embodiment, the disclosure provides a dermal filler apparatus, including: a barrel having a proximal end and a distal end; a plunger sliding within a portion of the body; a plunger handle mounted to the plunger; a needle extending from the distal end of the barrel; a light source extending from the distal end of the barrel; an optical detector extending from the distal end of the barrel; an indicator; and a processor.

In still another embodiment, the disclosure provides a self-contained dermal filler syringe apparatus, including: a body; a plunger sliding within a portion of the body; a plunger handle mounted to the plunger; a needle interchangeably coupled to the body; a light source extending from the body, the light source comprising a 568 nm light emitting diode (LED); a needle interchangeably coupled to the body; the needle including: a needle wall defining a conduit; a lighting wire electrically connected to the light source; a photodiode detector; an indicator in communication with the photodiode detector; and a processor in communication with the light source, the photodiode detector and the indicator.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a block diagram of the major components of the vascular detection sensor and their optical, signal, and power connections for various embodiments of the disclosure.

FIG. 2 shows a perspective view of a filler syringe inserted into tissue in accordance with one or more embodiments, where the optical sensor assembly is located in the hub of the needle.

FIG. 3 shows a cross-section of the hub region 200 of FIG. 2.

FIG. 4 shows a top-down view of a printed circuit board (PCB) and placement of mounted components such as that shown in FIG. 3.

FIG. 5 shows an arrange of the source and detector in the needle hub with conceivable optical elements positioned between the respective waveguides according to some embodiments.

FIG. 6 shows a cutaway view of the flange region 201 of FIG. 2 according to some embodiments.

FIG. 7 shows a perspective view of a filler syringe inserted into tissue in accordance with one of more embodiments, where the optical sensor assembly is located in the flange of the needle.

FIG. 8 shows a cross section view of the needle hub 708 of FIG. 7.

FIG. 9 shows a cutaway view of the flange region 700 in FIG. 7 according to certain embodiments.

FIG. 10 shows an embodiment in which the injection system is used to provide guided cannula placement.

FIG. 11 shows cross-sectional views through embodiments of a needle in which the source and detector waveguides are located close together (left) or further apart (right).

FIG. 12 shows a cross-sectional view through an embodiment of a needle with an interior channel for placement of waveguides that is constructed using a number of concentrically arranged hypodermic tubes.

FIG. 13 shows a cross-sectional view through another embodiment of a needle with an interior channel for placement of waveguides accepting multiple detector waveguides that is constructed using a number of concentrically arranged hypodermic tubes.

FIG. 14 shows a cross-sectional view through yet another embodiment of a needle that is constructed by forming a groove along the length of the needle to accommodate one or more waveguides and which is surrounded by a concentrically arranged hypodermic tube.

FIG. 15 shows a cross-sectional view through still other another embodiments of needles that are constructed by forming grooves along the length of the needle to accommodate one or more waveguides and which are each surrounded by a concentrically arranged hypodermic tube.

FIG. 16 shows return signal intensity levels for a fiber-containing needle device that is inserted into different tissues.

### DETAILED DESCRIPTION

In accordance with some embodiments of the disclosed subject matter, mechanisms (which can include systems, methods, and apparatus) for improved procedures for subdermal injections, including injections of dermal filler material, are provided.

Embodiments of the disclosure enable real-time detection of various types of tissues, including venous structures, in vivo. In various embodiments the disclosed device may enhance patient safety of a routine procedure that is widely practiced, including for example in cosmetic dermatology. In this procedure, cosmetic soft-tissue fillers are substances injected into the face and elsewhere in the body to replace volume loss that occurs with aging. A potential complication in this procedure is the inadvertent injection of the filler into a blood vessel, especially an artery.

Indeed, injection of injection material such as cosmetic dermal fillers in the facial area may result in unintended puncture of underlying vasculature and subsequent deposition of material into a blood vessel resulting in potentially serious complications to the patient. Soft tissue fillers also referred to as “dermal filler,” “injectable implants” or “injectables” are substances that are injected beneath the skin at different depths to reduce facial wrinkles and augment appearance that occurs due to loss through aging or injury.

Optical sensing of the tissue surrounding the distal end of the hypodermic needle may be used as the basis for informing trained medical personnel of a potential vascular puncture. The term medical personnel is meant to include dermatologists and plastic surgeons and is broadened to include among others, physicians, nurses, emergency medical technicians, and those that receive some degree of medical training as part of their jobs such as military personnel. Arteries and veins transport whole blood in the body and whole blood has the highest concentration of hemoglobin of any tissue. Furthermore, hemoglobin preferentially absorbs certain wavelength regions of light more strongly than other wavelength regions. In various embodiments, the apparatus and methods disclosed herein can be used to detect and avoid blood vessels when performing injections of certain materials such as dermal fillers; avoiding blood vessels when injecting other medications such as lidocaine; aiding in confirming vascular access in situations such as on the military field and in urgent situations such as cardiac catherization; aiding in confirming vascular access in patients with conditions such as peripheral vascular disease or diabetes; aiding in confirming vascular access to decrease risk of infusion injury or extravasation injury; aiding in confirming vascular access for use in chemotherapy infusions and for use with hyperosmolar medications that can be toxic to the tissue around the vessel; and/or aiding in confirming vascular access for sclerotherapy procedures.

In addition to absorption of light, biological tissues also scatter light and, similar to absorption, the amount and angular distribution of that scattered light is dependent upon the type of tissue. The combination of wavelength-dependent absorption and scattering coefficients, taken together as a tissue's optical properties, determines the optical transport properties, (i.e. the amount of light transported through a tissue). Absorption and scattering coefficients for tissues typical have a range of values. One important estimated property is the optical penetration depth, which estimates the ‘sensing’ distance that light can diffuse from a source and beyond which the ability to sense the changes in the tissue environment is limited. Furthermore, these optical properties also govern the amount of back-reflected light from different tissue types. Thus, the optical properties of blood and other tissues can be used to provide an optical detection-based injection system which makes injections safer by confirming prior to injection that the tip of the injection device has been correctly placed (e.g. that a needle being used for delivering an injection material such as a dermal filler is not located in a blood vessel).

In various embodiments the optical detection-based injection system can be incorporated in a standard hypodermic needle in such a manner that it does not significantly disrupt clinical practice while alerting medical personnel to the presence of vascular access. The design provides minimal disturbance to the present ergonomics of a standard injection system which includes a syringe body and hypodermic needle. Specifically, in certain embodiments the entire optical sensor assembly is incorporated into the parts of a standard injection system as a self-contained, standalone device without the need for electrical cables or optical waveguides to connect to external devices. Inner and outer needle diameters should not significantly change relative to currently-used sizes in order to maintain ease of physician use and patient comfort. The modular system allows rapid exchange of different needle gauges upon the same syringe body.

In general, an injection device according to embodiments of the present disclosure incorporates an optical sensor located at the tip of the hypodermic needle to detect vascular penetration during the placement of a hypodermic needle, alerting the user through a sensory signal to the presence of whole blood in the immediate tissue environment. The sensor system discriminates between whole blood and other tissue types by wavelength-dependent differences in the optical absorption and scattering of tissues, among others whole blood, thus providing the necessary feedback for safe hypodermic needle placement prior to injection.

As the sensor-enabled needle enters the tissue, light from the sensor will illuminate various tissue types found within the skin along the path of the needle to the intended placement. Propagation of light through different tissue types depends upon the wavelength-dependent optical absorption and scattering properties that are intrinsic to that tissue type. The optical signal is generated by detecting the fraction of the emitted light intensity that is back-reflected from tissue structures and collected by an optical waveguide. This backscattered light, which carries information about the immediate tissue type surrounding the hypodermic needle, will be guided to an optical detector that generates an electrical signal that can be analyzed. When the sensor-enabled needle is located in tissue with low optical absorption, for example due to a low concentration of hemoglobin, the intensity of scattered light will be high resulting in a high optical signal being detected. In contrast, when the sensor-enabled needle is located in a blood vessel, the emitted light will be strongly absorbed by hemoglobin producing minimal backscattered light and minimal optical signal. Since a low optical signal is detected, the logic circuit enables a visual and or audible signal alerting the trained medical staff to the possibility of vascular puncture.

The arrangement of components around a standard barrel syringe typically used for soft tissue filler procedures has been designed to be ergonomic. Since multiple approach angles are often used in filler procedures, components for embodiments of the disclosed injection system are built directly into the standard needle components to provide the physician with a clear, unobstructed view of the tissue, providing a comparable feel to traditional filler syringes and freedom of movement without wired connections. The device has been designed so that the tubular fluid delivery elements (e.g. needles and cannulas of different gauges) can be easily exchanged in order to inject different types of materials (e.g. having different viscosities) into different types of tissues through a rapid exchange connection making the exchange procedure simple and repeatable.

Embodiments disclosed herein include a needle assembly which improves the safety of subdermal injections of cosmetic fillers by detecting whether the needle tip is located in a blood vessel or other type of tissue. Furthermore, the procedures have implications beyond this specific function: for example, the apparatus and procedures can be used in certain embodiments to ensure that medications are safely injected into the vascular system by detecting blood near the needle tip prior to injection. Embodiments of the device can also be used to monitor the perforation of blood vessels. An advantage of the device is that the detection of vasculature penetration by a hypodermic needle can be accomplished in real time using an inexpensive, disposable system having a minimal physical footprint. Other advantages include the fact that the device requires little training to use, does not change the current practices of placing filler, and does not add time to the procedure. This device could be used in medical applications in both clinical environments (including human and veterinary clinical settings) and field setting by military personnel and first responders.

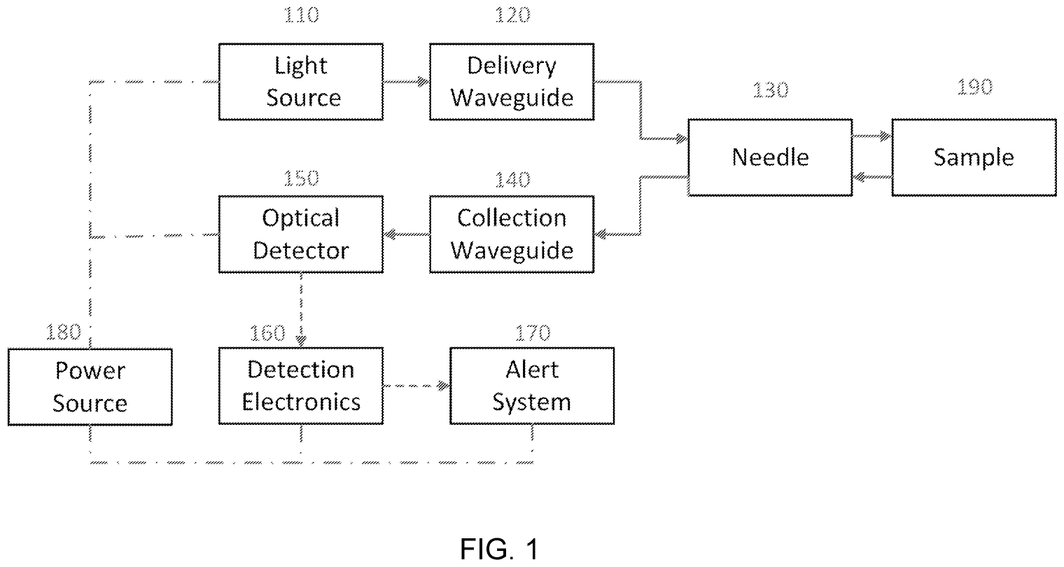

A block diagram of an injection device that is capable of vascular detection is presented in FIG. 1, in which optical pathways are indicated by solid lines with arrow heads, electronic signals are indicated by small dashed lines with arrow heads, and electronic power is indicated by larger dashed lines without arrow heads. The main components of the sensor-enabled injection device are a light source [110], delivery waveguide [120], collection waveguide [130], hypodermic needle assembly with embedded sensor [140], optical detector [150], electronics [160], sensory alerting system [170], and power source [180]. Light generated by the light source [110] is directed into a delivery waveguide [120], which transports light along the shaft of the hypodermic needle [130] to the tip of the bevel on the needle and into a sample [190] (e.g. a tissue). Reflected light is collected by the waveguide [130] and transmitted to the optical detector [150], which converts the optical signal into a first electrical signal. The electrical signal is fed into an electronics [160] that analyzes the first signal for tissue type and sends a second signal to a sensory alerting system [170]. Electrical power to the light source [110], optical detector [150], analysis electronics [160] and alter system [170] is supplied by a common power source [180]. Each of the components is described in further detail below.

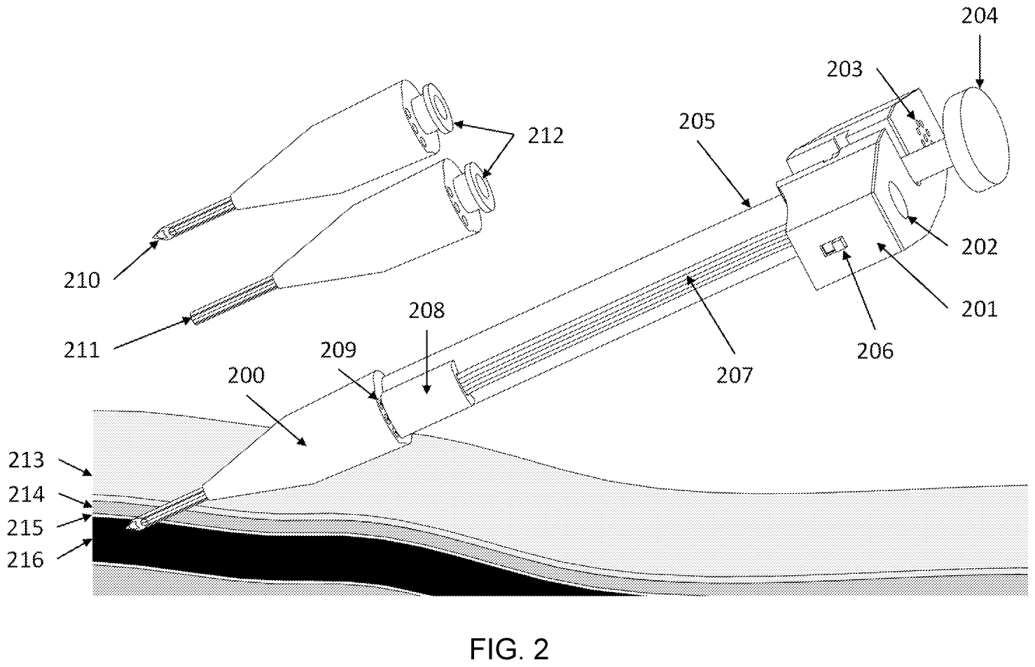

In general, a standard syringe used in medical practice including the application of dermatological fillers, has an extended tubular barrel that is terminated at the distal end with a needle adapter, often of the Luer lock form, and the proximal end of the barrel has a flange. A piston or plunger creates a tight seal within the barrel and either expels fluid out or suctions fluid in. The needle includes a shaft, typically made of stainless steel, whose proximal end is terminated in the needle hub. The needle hub is outfitted with an adapter, typically of the Luer lock form, that connects it to the barrel via twist-lock or compressive friction fit. The shaft contains an open central lumen used to transport fluid. The proximal end is sealed within the needle hub and the distal end is terminated with a bevel that forms an acute angle with the shaft such that it forms a point. The preferable bevel angle is approximately 20 degrees, although other bevel angles are possible. In various embodiments, the needle can be a needle having a sharpened, beveled end with an opening for fluid delivery at the very end as shown in FIG. 2. In some embodiments, the needle can have an opening for fluid delivery that is not at the end but instead is on the side of the needle shaft near the end. In other embodiments, the needle can have a blunted or rounded end rather than a sharpened or beveled end. In one particular embodiment the needle can have a blunted or rounded end as well as a fluid delivery opening that is on the side of the needle shaft near the end (e.g. TSK STERiGLIDE Premium Aesthetic Dermal Filling Cannula from Precise Medical).

The arrangement of components for the injection system described in FIG. 1 is shown in FIG. 2. A syringe in accordance with one or more embodiments of the disclosure includes a piston or plunger [204] disposed in the barrel to expel the filler or other material, barrel [205] with finger flanges, and a hypodermic needle attached to and projecting from the hub [200] of the needle.

In one embodiment, elements of the optical sensor are located within a custom needle hub [200] located at the distal most portion of the device. Power, logic, and alert systems are located within a custom flange [201]. Sensory alerts may include visual [202] and auditory [203] alerts. The flange [201] is coupled to a sterile and preassembled piston [204] and barrel [205], providing ergonomic finger holds for the user. The barrel may be preloaded with a medical substance, in particular a soft tissue filler. Optional enable mechanisms [206] (depicted as a switch, although other mechanisms could be used) give the user control over device functionality. Electrical wires [207] are run from the flange along the outside of the barrel to a quick-connect electrical harness [208]. Techniques such as providing spring-loaded pins and oversized pads [209] may be used to facilitate the electrical connection between the quick connect harness and the hub. The electrical wires carry power from the flange to the hub to turn on the light source and optical detector, which are housed within the hub.

Additional electrical wires also transmit detector signals to detector logic electronics located in the custom flange for analysis and alert. The needle hub [201] is removable throughout a procedure and can be exchanged between different types of needles, e.g. sharp tip needle [210], blunt tip needle [211], or a different gauged needle, via mechanisms like twist or compression fittings [212]. Each hub contains a custom needle shaft that incorporates a source waveguide and detector waveguide. The waveguides are aligned optically within the hub to their respective optical elements. As the device passes through tissues such as dermis [213] and muscle [214], light from the source is passed through the source waveguide into the tissue. Reflected light is collected by the detector waveguide and sent to the detector. If the device approaches a vessel wall [215] and if it eventually enters whole blood [216], the logic electronics within the flange detects a change in the optical signal level (i.e., the reduced light at the detector) and properly alerts the user.

The standard barrel syringe typically used for soft tissue filler procedures has been designed to be ergonomic. It is designed to enable the physician to use it from a variety of angles, which can be important in procedures such as injection of dermal filler material. The device has been designed so that the tubular fluid delivery elements (e.g. needles and cannulas of different gauges) can be easily exchanged in order to inject different types of materials (e.g. having different viscosities) into different types of tissues. In certain embodiments, a new hub with an attached tubular fluid delivery element may be attached to the device; in embodiments such as this, the exchange of the tubular element along with the hub only requires re-establishing the electrical connections but not optical connections (e.g. connections between the fibers and the light source and/or detector) since the light source and detector are contained with the hub, making the exchange procedure simpler and more reliable.

In some embodiments, the hub is coupled to the electronics unit by one or more connections that permit the electronics unit to control and optionally power the light source and detector and to obtain data from the hub. The control connections between the hub and the electronics unit may be made wirelessly (e.g. using Bluetooth) or may be made via electrical contacts that can be readily connected together when the hub is joined to the upper portionof the device (e.g. to the syringe barrel and electronics unit as shown in FIG. 2) using a connector assembly as shown in FIG. 2. In some embodiments the electrical contacts may be made by abutting conductive pads on the hub with complementary conductive pads on the upper portion of the syringe, and in other embodiments the connections may be made by plugging conductive leads into complementary receptacles.

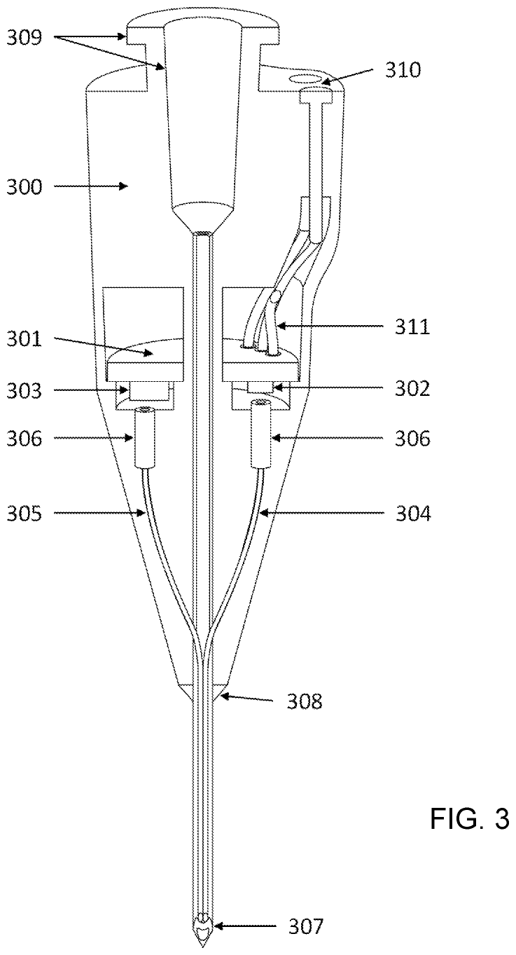

In one embodiment, the optical sensor elements are located in a custom needle hub [300] may be arranged as shown a cross-sectional view in FIG. 3. Inside the hub is an annular printed circuit board (PCB) [301] which incorporates electrical connections and mounts a light source [302] and an optical detector [303] as well as other passive electronics. A waveguide [304] carries light from the source to the distal tip of the device. Another waveguide [305] carries reflected light from the tissue back to the detector. Both waveguides may be aligned within the device using alignment tubes [306]. Both waveguides exit the hub and are integrated with a custom needle [307] that delivers and receives the light into the tissue. The arrangement of the needle shaft [308] relative to the PCB and waveguides is also shown in FIG. 3. The needle hub connects to the syringe using twist or compression fittings [309]. Power for the hub and data from the detector are sent to a processor via electrical pads [310]. Wires [311] connect these pads to the PCB.

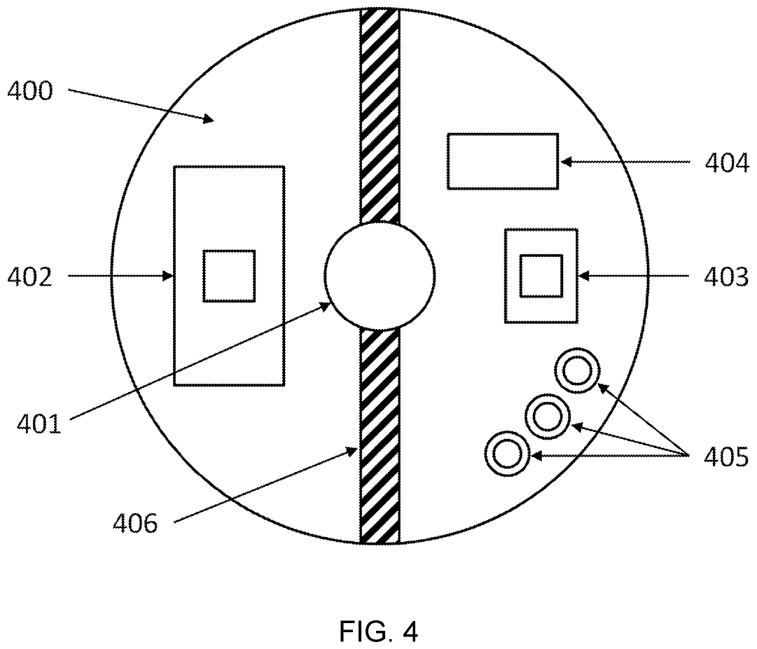

In one embodiment, the arrangement of optical and electrical components located on an PCB [400] is shown in a top-down view in FIG. 4. In some embodiments, a preferred shape of the PCB is annular to maximize use of space in the needle hub and allow an opening for passage of the needle shaft [401]. Coupled (e.g. via soldering) to the PCB is a light source [402] and optical detector [403] as well as supporting electrical components [404]. Power for the components comes from an external source located in the custom flange and enters the board through power pads [405]. Data from the detector is also sent to the flange via these power pads. Optical isolation between the source and detector units is ensured through the use of a keep-out zone [406] and may reduce detection background.

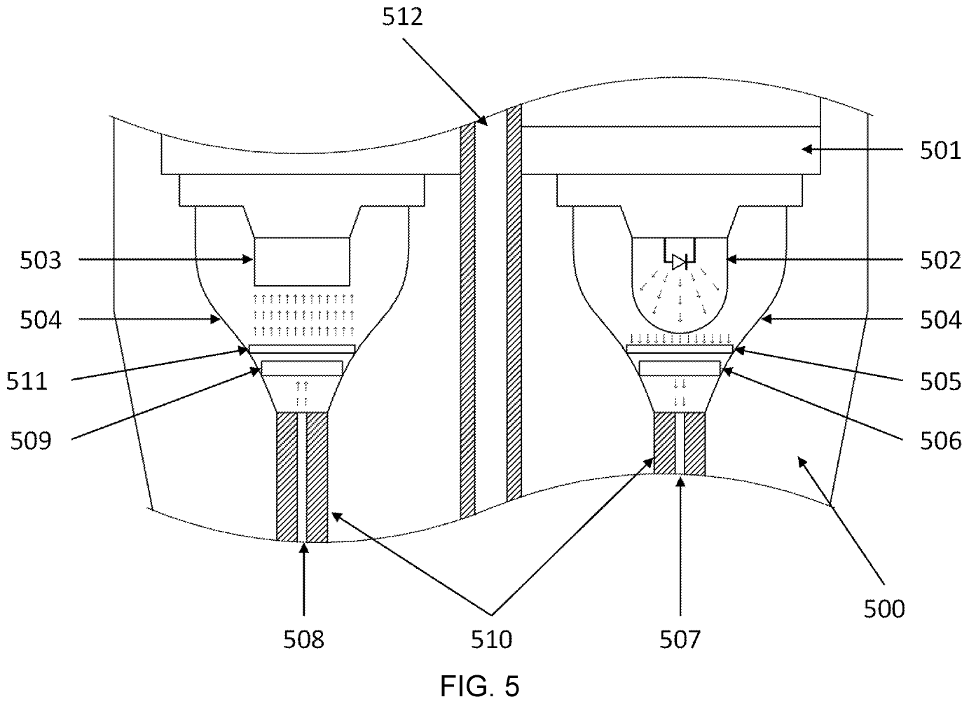

The interface between the optical source, optical detector, and respective waveguides may be made in many ways. In one embodiment, the source [502] and detector [503] are installed on a custom PCB [501] as shown in FIG. 5. The custom hub [500] positions each optical element within its own isolated compartment formed within the needle hub. In some embodiments, the surfaces [504] of these compartments may be coated with reflective materials to maximize light transmission and collection. In some embodiments, the light source unit may also contain an optical transmission filter [505] to remove wavelengths from the source light that are undesirable, enabling a broader range of light sources with varying spectral purity to be used. In some embodiments, an optical element, for example a lens [506] may also be used to focus the source light into the source waveguide [507]. In further embodiments, the source waveguide may be aligned to the hub using an alignment tube [510]. Reflected light that is collected from the sample by the device travels through the detector waveguide [508] which is aligned to the hub using an alignment tube. In some embodiments, the light leaving the detector waveguide can be focused using an optical lens [509] to illuminate the detector surface. In some embodiments, an optical filter [511] can be used between the detection waveguide and detector to remove wavelengths of light not sent by the device such as ambient room light, directed procedure lights or operating room lights.

The optical sensor assembly in FIG. 5 includes a light source, waveguides, and optical detector. Numerous devices can be used as a light source [502] including, but not limited to, a light emitting diode (LED), a superluminescent diode (SLD), a laser, or a diode laser among other coherent and incoherent sources. In preferred embodiments, the light source is a compact LED with an optical power that shall not exceed that of a Class I laser device in this wavelength range (as per 21 CFR 1040.10). In various embodiments, the light source provides visible light, e.g. light having a wavelength in a range of 400 nm-700 nm. In certain embodiments, a preferred wavelength range of the illuminating light contains wavelengths in the 400-600 nm spectral region because this spectral region encompasses the major hemoglobin absorption features. Spectral bands of undetermined width within the 400-600 nm range can be used to detect the presence of hemoglobin. In certain embodiments, a preferred range of usable wavelengths is between 500-600 nm may provide increased sensitivity because there are fewer biological absorbers in this region. In further embodiments, a narrower spectral region incorporating the wavelength range between 568-577 nm is preferred since there is a significant difference in the optical absorption coefficients between hemoglobin, the primary absorber in whole blood, and myoglobin, the major absorber in muscle tissue. Confinement of the illuminating light to the 568-577 spectral region is expected to increase the sensitivity of the device and better discriminate between blood and muscle. Nevertheless, light with other wavelengths can also be used and are encompassed by the present disclosure.

Light generated by commercial light sources may extend beyond the intended spectral regions. In a preferred embodiment, emission from light sources [502] may be spectrally filtered using a wavelength-dependent optical component, such as an inline optical transmission filter, to select the optimal wavelength region or regions that enhance the detection of the target tissue. In the case of detecting vascular puncture, the optical transmission filter will have high wavelength-dependent transmission in one or more spectral regions corresponding to optical absorption features of hemoglobin and low transmission in regions with low hemoglobin absorption. Wavelength selection may be based on increasing the overall sensitivity to whole blood relative to other tissue types.

The optical detector unit may include a photodetector, amplifier circuit, and optical filter. A preferred embodiment for the photodetector is a single pixel photodiode selected for a high quantum efficiency over the wavelength range of the illumination band. In other embodiments, multiple detectors or a single detector with a plurality of pixel sensors may be used. An optical filter may be used to reject unwanted wavelength regions produced by the light source such that the sensitivity for detection of hemoglobin is increased, or to reject undesirable stray light collected from external sources such as operating room lights. In another embodiment, wavelength dispersive element, such as a prism, grating or grism, may be incorporated.

Waveguides are optical structures that are used to transmit light from the light source across the needle assembly, in particular the needle shaft, to the sample and back-scattered light from the tissue to the optical detector. Waveguide structures applicable for various embodiments of the disclosure include a combination of, but are not limited to, silica/silica optical fibers, silica core/polymer cladding optical fibers, polymer/polymer optical fibers, or hollow tubes. Further, in some embodiments light may be transmitted through the needle shaft and into the tissue and reflected light may be collected via the waveguide/optical fiber associated with the needle. In certain embodiments, waveguides, in particular optical fibers of various compositions, may have a range of core diameters of up to 500 microns and more particularly between 50 microns and 200 microns, and range of numerical apertures (NA) between 0.2 and 0.5 although other diameters and NAs are also possible. A preferred optical fiber has a small optical cladding and jacket thickness to reduce the amount of space taken up by the fiber. The hypodermic needle may incorporate a single waveguide that transceives both illumination and back-scattered light, or may incorporate a plurality of separate waveguides for carrying illumination and backscattered light. Delivery and collection waveguides may have the same core diameter and NA or they may have different combinations. In one embodiment, silica/silica optical fibers are a preferred material for delivery and collection waveguides, having a core diameter of 100 microns and an NA of 0.22. This combination of NA and core diameter provides a confined illumination beam and large collection area to collect the back-reflected light.

In an alternative embodiment, a single multimode fiber can be used to transmit light from the source to the tissue and back from the tissue to the detector, although there are some challenges to this approach due to potential cross talk of light within a single waveguide, whereas the use of two fibers limits cross talk. Nevertheless, in some embodiments a single waveguide may be used, preferably a multimode fiber with a small cladding and thin jacket, to deliver and collect the back scattered light from the tissue, which allows the optical sensor at the needle tip to have a minimal cross-sectional footprint. In this embodiment, the forward traveling illumination light propagates along the waveguide until it reaches the tissue/fiber interface. In a preferred embodiment, it is noted that a single fiber that transceives the light may also include small scattering centers or defects within the fiber core that may also reflect light back through the fiber to the detector that does not reach the tissue. The ratio of collected light generated by scattering in the fiber to the diffusely collected light scattered in tissue is referred to as crosstalk. The magnitude of these sources of backscattered or reflected light may be significantly higher than the magnitude of backscattered light from the tissue, which presents a further difficulty in this embodiment.

In one embodiment, a solution to avoid cross-talk in the single fiber is to insert a polarizer or polarizing element placed at the tip of the fiber that weights the detected light towards light that has scattered in the tissue, which would improve optically-detected signal by reducing the overall amount of crosstalk contained within the signal and facilitate the use of only one fiber.

In another embodiment which uses only a single fiber for both light transmission and data collection, a dual clad fiber, also referred to as double clad fiber, may be used. In the dual clad fiber the inner core (˜6-10 μm) transmits the illumination light and the collected tissue light is collected by the inner cladding. The illumination and detection light is physically separated within the fiber by structures built into the fiber. Physical separation between the internal reflected light and collected tissue scattering may be achieved by use of a dual-clad fiber coupler or by physically blocking the image of the core projected on to the detector. The degree of cross talk between the core and inner cladding is predominantly a function of the ability to couple the illumination into the core only.

In yet another embodiment, an air or liquid-filled tube can be used as a waveguide where the liquid is transparent to the transmitted wavelength of light and the wall of the waveguide is reflective, providing acceptable light transmission.

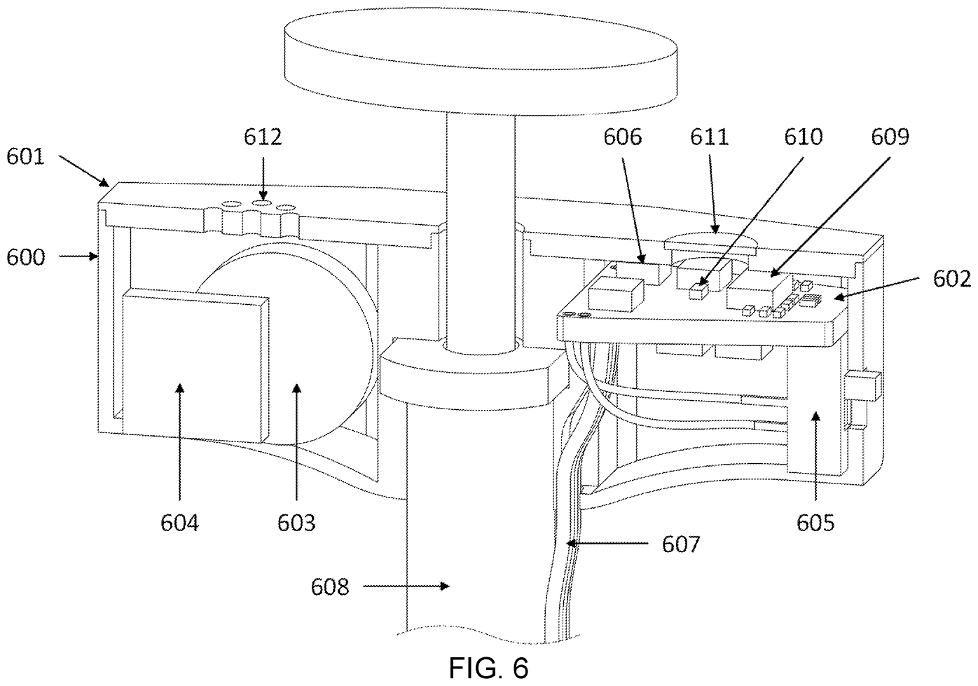

In a preferred embodiment where the optical sensor elements are located within the hub, the power, processing, and alert systems are found within the flange. A cut-away illustration of this embodiment is found FIG. 6 where a custom flange including a main body [600] and cover [601] protects and aligns all electrical elements. Within the main body is a control PCB [602], battery [603], auditory alert component [604], and operator control such as a toggle or momentary switch [605]. The control PCB receives power from the battery and uses a regulator [606] to power on-board components. Electrical wires [607] attached to the outer wall of the barrel [608] send power to the hub and receive data from the hub-based detector. Detector data is processed by on-board logic [609]. The control PCB automatically alerts the user to pre-determined levels of reflection via a visual indicator [610] and/or an auditory alert. The cover of the flange contains a window [611] to transmit the visual alert as well as provisions for audio transmission [612].

The on-board logic unit [609] may use either an analog- or a digital-based comparator electronics circuit that registers the immediate electrical signal converted from the optical backscattered light against a predetermined threshold signal. If the electronic signal is high, then the comparator circuit determines that the needle is in non-target tissue (i.e. hemoglobin-rich tissue such as a blood vessel) and outputs a low signal. If the signal is low relative to the predetermined threshold, the sensory-enhanced needle is determined to be surrounded by the target tissue, more specifically blood, and the circuit therefore outputs a high signal to drive an audible or visible alert. In some embodiments of the on-board logic unit, an integrator or moving average circuit may be implemented to smooth out noise in the real-time data and may facilitate identification of threshold levels or reference values. Additional ‘smart’ algorithms may be established to implement the use of “warning” and “stop” signals. This threshold signaling can be further optimized to minimize false alarms to provide a “warning” signal when the fiber ends and needle tip are close to, but not inside of, a structure such as a blood vessel as opposed to only being able to provide a “stop” signal after the needle has entered a particular tissue type.

While embodiments of the device using threshold signal levels are suitable for dermal filler applications, modification of the optical sensor components and signal processing procedures may be required in other embodiments, for example for other applications involving identification of other tissue types or anatomical locations that are not blood vessels or blood. Since a tissue type may be identified based the wavelength-dependent absorption and scattering properties of the target tissue, the intensity of the backscattered light may provide a sufficient information for needle guidance and/or placement in addition to needle detection. In certain embodiments, the apparatus and procedures may be used for the identification of tissues or anatomical locations, in particular compartments containing fluids that are not necessarily blood such as synovial and spinal fluids which may be drawn into a syringe. These embodiments may be further enabled by the acquisition of more information from a set of collection waveguides distributed at different distances from the illumination waveguide, where the distance is defined as the center-to-center distance between these waveguides.

In some embodiments, a transmitter, for example a Bluetooth transmitter, may be incorporated into the custom flange to enable signal data to be transmitted from the injector device (e.g. wirelessly) to a computing system for display in real time or at a later point in time. In various embodiments, the threshold or cutoff levels may be established using one or both of ex vivo and in vivo experimental findings.

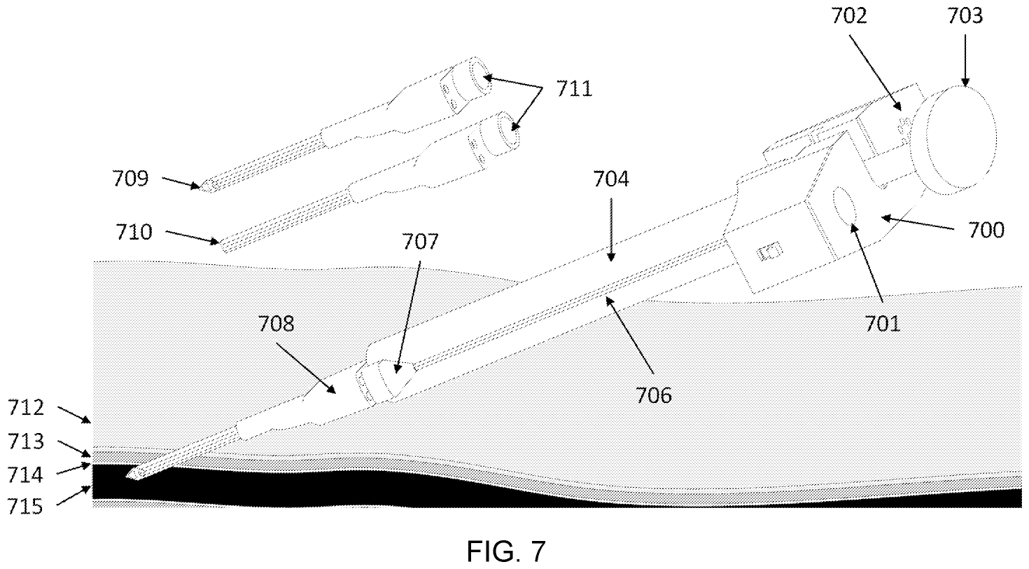

A second arrangement may exist where the optical sensor components are incorporated into the modified flange [700] along with the power, logic, and alert systems as shown in FIG. 7. Alerts can include visual [701] and auditory [702] alerts. The flange slides over a sterile and preassembled piston [703] and barrel [704], providing finger holds for the user. Optional enable mechanisms [705] give the user control over device functionality. Optical waveguides [706] run from the flange along the outside of the barrel to a quick connect optical harness [707]. Techniques such as butt-coupling and face alignment [708] may be used to facilitate optical connection between the quick connect harness and the needle hub. The needle hub is removable throughout a procedure and can be exchanged between types such as a sharp tip needle [709] and a blunt tip needle [710] via mechanisms like twist or compression fittings [711]. Each hub contains a custom needle that incorporates source and collection waveguides, where the waveguides are aligned optically to the quick connect harness which in turn aligns them to their respective optical elements within the flange. As the device passes through dermis [712] and muscle [713], light from the source is passed through the source waveguide into the tissue and reflected light is collected by the detector waveguide and sent to the detector. As the device approaches a vessel wall [714], and may eventually enter whole blood [715], the logic within the flange detects the reduced light at the detector and properly alerts the user.

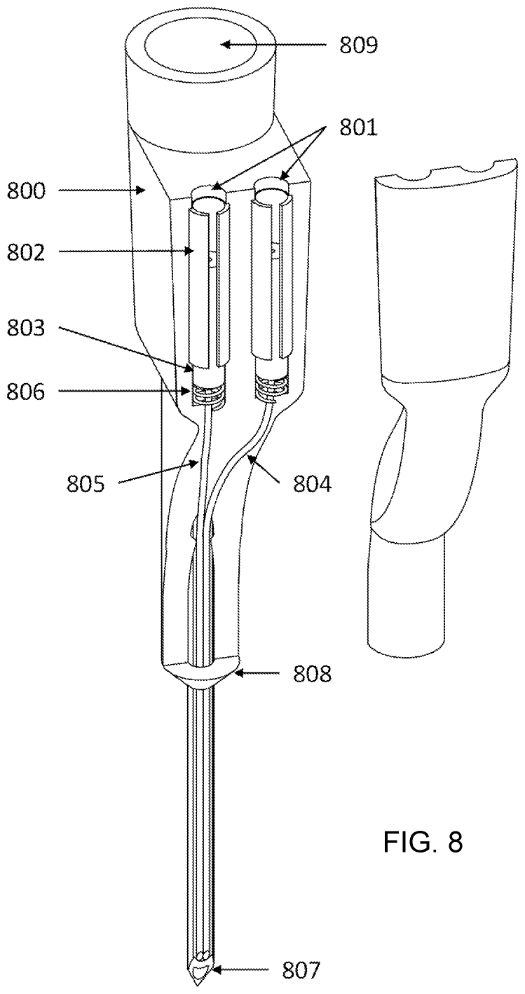

Furthermore, in the second arrangement described in FIG. 7, light is transceived between the modified flange [700] and the tip of the needle shaft by coupling of the waveguides, in particular optical fibers, through a rapid exchange optical interface. One embodiment of the rapid exchange optical interface is provided in FIG. 8. Thus, in the second arrangement only passive optical elements are located within the custom needle hub [800]. This needle hub is aligned optically to waveguides extending from a custom sensory and alert flange. The alignment occurs via optical landing pads [801]. These landing pads have alignment tubes [802] that contain a source waveguide [803] and detector waveguide(s) [804], in the case of a plurality of waveguides. The source waveguide carries light from the flange to the distal tip of the needle. The detector waveguide(s) carries reflected light from the tissue back to the landing pads where it can be transmitted to the flange. Both waveguides exit the hub and are incorporated into a custom needle [805] that delivers and receives the light into the tissue. The needle can be held to the hub using adhesive, e.g. a biocompatible epoxy [806]. The hub can connect to the syringe using twist or compression fittings [807].

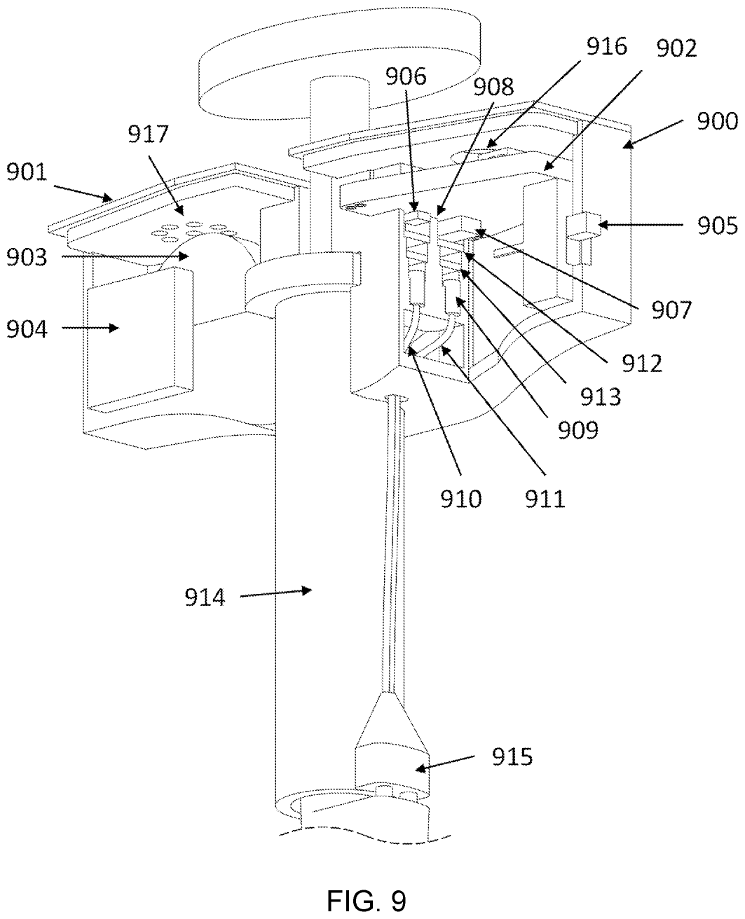

In one embodiment, the active optical elements, power electronics, logic, and alert systems can be located within the flange (FIG. 9). A custom flange including a main body [900] and cover [901] protects and aligns all elements. Within the main body is a control PCB [902], battery [903], auditory alert component [904], and operator control such as a toggle or momentary switch [905]. The control PCB receives power from the battery and uses a regulator [906] to power on-board components including a source [907], detector [908], and logic elements [909]. A physical barrier in the flange geometry between source and detector [910] prevents cross-talk between elements. An alignment tube [911] positions a source waveguide [912] under the source. Another alignment tube positions a detector waveguide [913] under the detector. Optional filters [914] and lensing elements [915] can be placed between the source, detector, and respective waveguides in order to maximize light delivered or received, or to take advantage of less expensive and/or broadband sources or to remove background illumination such as operating room lights. Both waveguides extend out of the main body of the flange and are attached to the outer wall of the barrel [916]. These waveguides eventually terminate in a quick connect optical harness that delivers light to and from the hub. Received light from the needle tip enters the flange through the detector waveguide where the detector and logic elements process the data on-board. The control PCB automatically alerts the user to pre-determined levels of reflection via a visual indicator [917] and/or an auditory alert. The cover of the flange contains a window [918] to transmit the visual alert and includes provisions for audio transmission [919].

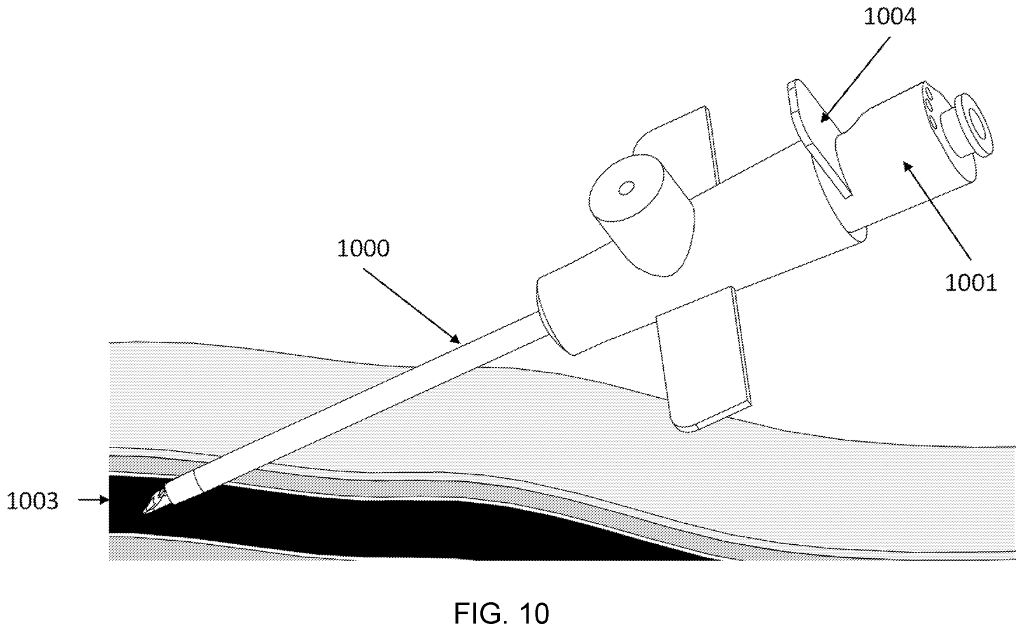

In another embodiment, the device can be used in conjunction with a standalone cannula for guided cannula placement (FIG. 10). In this configuration, the cannula [1000] slides over a custom needle hub [1001] before the needle is inserted into the body. Vascular detection from the needle allows for placement of cannula in bloodstream [1003]. After placement, the cannula can be fixed to the body and the custom needle hub removed using a removal tab for easy grip [1004].

Various embodiments of the injection device can include the use of small gauge needles appropriate for subdermal injections which are outfitted with waveguides, in particular optical fibers. In one preferred embodiment, one of the fibers is used to deliver light from the source to the tissue and the other fiber is used to collect light from the tissue and deliver it to the optical detector. Furthermore, the device may have interchangeable needles and cannulas, where the range of acceptable needle/cannular/tubular element gauges may be between 23 gauge and 30 gauge, and for some applications as large as 14 gauge. As an example of the size ranges, the minimum inner diameter is approximately 0.159 mm (the inner diameter of a 30 gauge needle) and the maximum outer diameter is approximately 0.641 mm (the outer diameter of a 23 gauge needle). Thus, there is balance between an inner diameter that is sufficiently large to accommodate flow of materials and an outer diameter that is sufficiently small to avoid tissue injury and provide optimum comfort to the patient receiving the injection. Incorporation of optical fibers within the wall or along the exterior of needle shaft places further constraints upon the design of a sensor-embedded needle for vascular detection.

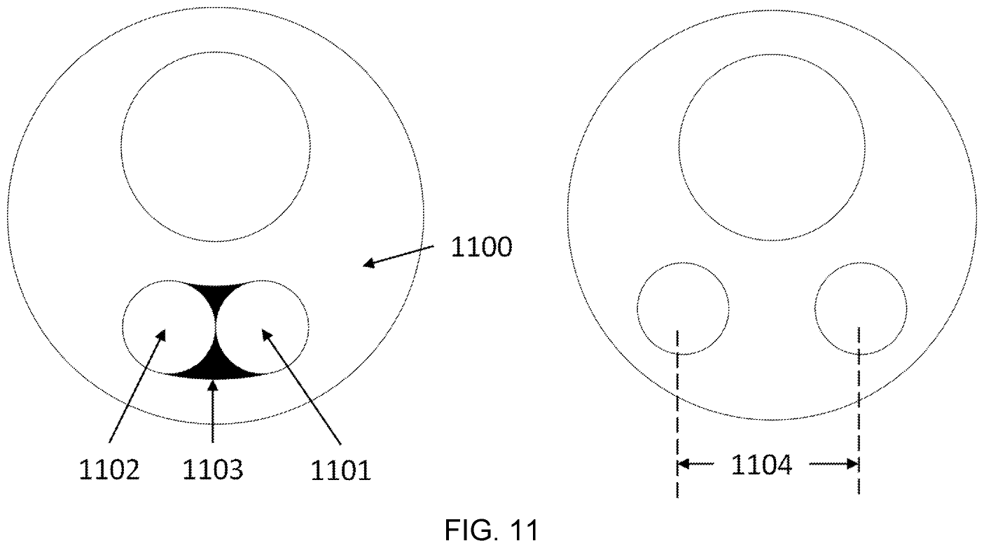

Various embodiments of a sensor-embedded needle are presented herein. In one embodiment of the custom needle, the source waveguide [1101] and detector waveguide [1102] are embedded within the wall of a custom extruded, asymmetric hypodermic tube [1100]. A cross-sectional image is shown in FIG. 11 of fibers positioned in contact (FIG. 11, left) and at a distance from one another (FIG. 11, right). The extrusion is designed to maximize the inner diameter size of the needle, which is used for fluid transfer, while minimizing the outer diameter size. The spacing between these channels (i.e. the openings within the hypodermic tube into which the waveguides are inserted) can be controlled to change the distance between waveguides [1104]. In one embodiment, a single channel with an oval-shaped cross-section is formed to accommodate both fibers abutting one another (FIG. 11, left) and in another embodiment two channels are formed, one for each fiber, spaced apart by a distance [1104]. Medical-grade epoxy [1103] can be used to fill gaps and secure all elements in place.

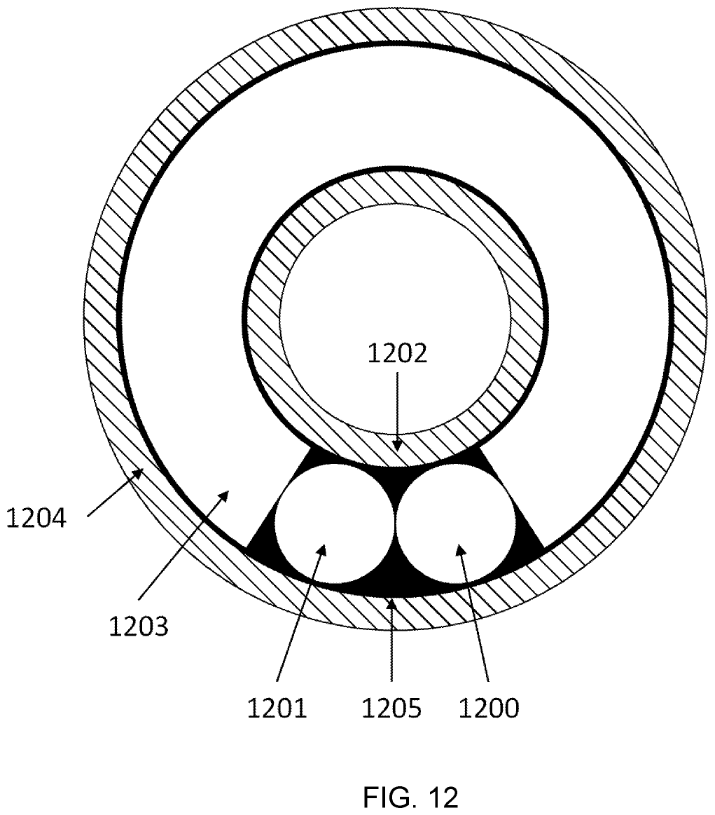

In one embodiment of the custom needle, the source waveguide [1200] and detector waveguide [1201] are embedded in an arrangement of concentric hypodermic tubes as shown in a cross-sectional image in FIG. 12. A thin-wall protective inner tube [1202] creates an inner lumen for fluid passage. A thick-wall hypodermic tube [1203] is cut along its length and the waveguides are placed in the opening. Finally, a second thin-wall protective outer tube [1204] is placed over the entire assembly. Medical-grade epoxy [1205] can be used to fill gaps and secure all elements in place.

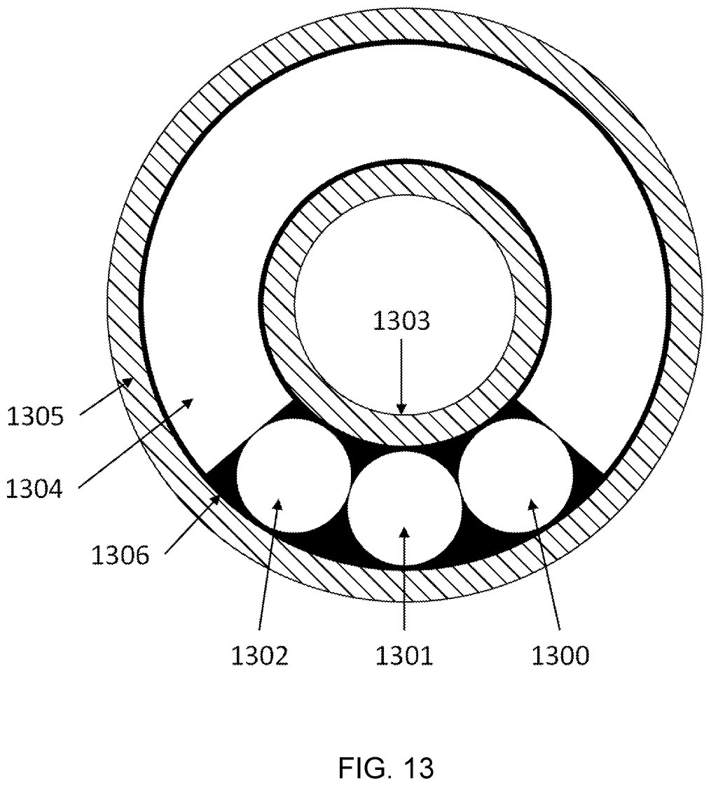

The arrangement of delivery and collection optical fibers, particularly the spacing between the ends, impacts the average distance at which the device can sense structures, such as vasculature. In general, a pair of delivery and collection fibers whose ends are very close or in contact with one another (e.g. see FIG. 11, left) may sense backscattered light from a change in tissue type or anatomical structure at a closer needle-tissue distance, providing a short warning distance, but with higher signal intensities. In contrast, a set of spatially-separated delivery-collection fibers (e.g. see FIG. 11, right) collects backscattered light at greater distance from the needle tip but at lower intensities. Thus in various embodiments, when the device senses a blood vessel at a closer distance a warning can be provided to the user that is akin to a “stop light” whereas if the device senses a blood vessel at a further distance a different warning can be provided which is akin a “yellow warning light”; the warnings can have different tones, volumes, patterns, brightness (in the case of a visual warning), or other readily-distinguishable differences to alert the user to the difference in distance from a blood vessel. In one embodiment of the custom needle, there can be multiple detector waveguides as illustrated in the cross-sectional image in FIG. 13. A close-range detection waveguide [1301] can be positioned next to the source waveguide [1300], while a far-range detection waveguide [1302] can be placed further from the source waveguide. All waveguides can be embedded in an arrangement of concentric hypodermic tubes, similar to the embodiment of FIG. 12. A thin-wall protective inner tube [1303] creates an inner lumen for fluid passage. A thick-wall hypodermic tube [1304] is cut along its length and the waveguides are placed in the opening. Finally, a second thin-wall protective outer tube [1305] is placed over the entire assembly. Medical-grade epoxy [1306] can be used to fill gaps and secure all elements in place.

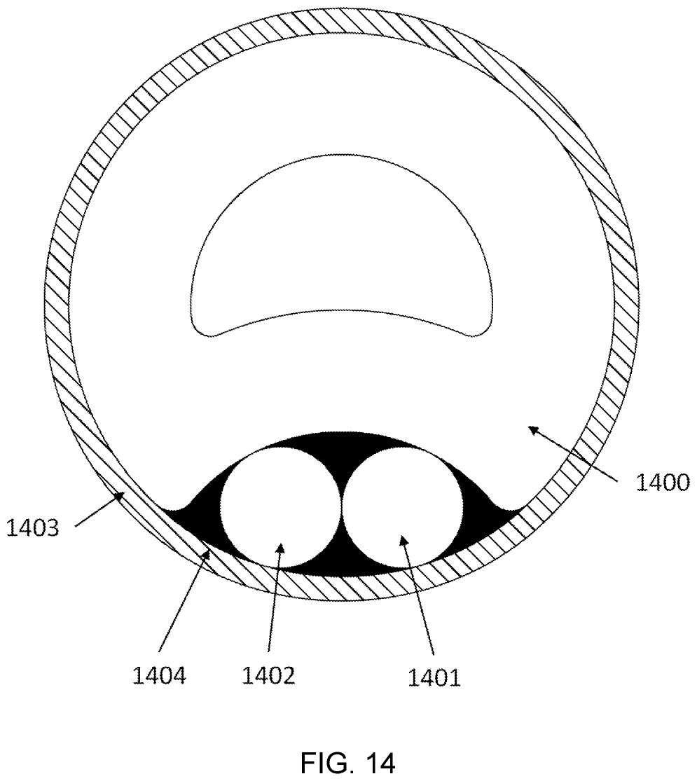

Smaller gauge needles have thinner walls that are comparable in thickness to optical fibers. For example, a 100 micron core optical fiber with polyimide jacket has an overall diameter of approximately 124 microns while a 30 gauge needle has a wall thickness of approximately 76 microns. To maintain the interior luminal diameter of a small gauge needle while slightly increasing the exterior diameter, optical fibers can be incorporated into the exterior of the needle shaft. Placing longitudinal track(s) along the needle shaft fixes the position of the optical fibers while reducing the overall diameter. In one embodiment of the custom needle, a hypodermic needle [1400] can be crimped or otherwise modified to create a groove where the source waveguide [1401] and detector waveguide [1402] can be placed as shown in FIG. 14. A protective thin-wall hypodermic tube [1403] can be placed around the assembly to protect the fibers. Medical-grade epoxy [1404] is used to fill gaps and secure all elements in place. As with other embodiments, multiple detector waveguides may be added along the outer diameter of the custom needle if desired.

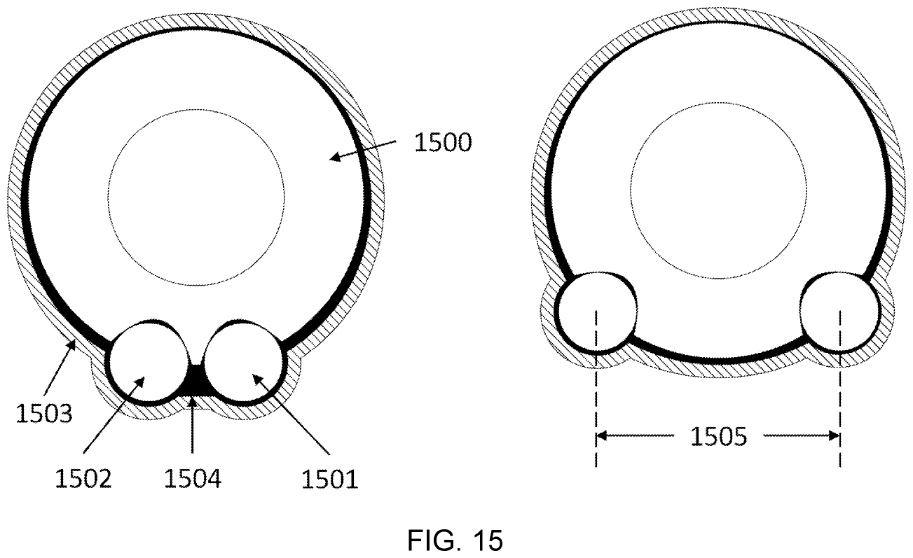

In yet another embodiment of the custom needle, a hypodermic needle [1500] can be etched to create individual grooves for the source waveguide [1501] and detector waveguide [1502] as shown in FIG. 15. Spacing of the grooves can be controlled to change the distance between waveguides so that the waveguides may be close together (e.g. FIG. 15, left) or spaced a distance [1505] apart (FIG. 15, right). A protective thin-wall hypodermic tube [1503] can be placed around the assembly to protect the fibers. Medical-grade epoxy [1504] can be used to fill gaps and fix all elements in place. As with other embodiments, multiple detector waveguides may be added along the outer diameter of the custom needle if desired.

In another embodiment, a power supply may be located in the needle hub. In some embodiments the electronics may be located primarily in the hub with the upper electronics unit being limited to housing mechanisms for indicators (e.g. audible or visual indicators) and/or a power switch. In this and other embodiments, the hub may communicate using a wired or wireless (e.g. Bluetooth) connection to the electronics unit to convey information to control features such as the visual and auditory warning signals.

The device has been designed to be ergonomic. It is designed to enable the physician to use it from a variety of angles, which can be important in procedures such as injection of dermal filler material. The device has been designed so that the tubular fluid delivery elements (e.g. needles and cannulas of different gauges) can be easily exchanged in order to inject different types of materials (e.g. having different viscosities) into different types of tissues. In certain embodiments, a new hub with an attached tubular fluid delivery element may be attached to the device; in embodiments such as this, the exchange of the tubular element along with the hub only requires re-establishing the electrical connections but not optical connections (e.g. connections between the fibers and the light source and/or detector), making the exchange procedure simpler and more reliable.

The device may provide auditory and/or visual feedback, generally emanating from the syringe handle where it can be readily visualized and/or heard by the person performing the injection. This audible and visual feedback from the handle provides an improvement relative to having to watch the end of the needle, which can be hard to see and may be obscured by parts of the syringe, including the barrel or handle. In contrast, according to current clinical best practice procedures, the person performing the injection has to closely watch the needle so that any blood reflux will not be missed, whereas the automatic blood detection provided by the present device eliminates the need to directly observe the needle.

The underlying operative principle of the device is that the system identifies spectral differences between tissue types to detect vasculature penetration during the placement of a hypodermic needle and prior to injection. The system detects and identifies differences in optical absorption between blood and surrounding tissues to help guide the hypodermic needle to a safe placement location prior to injection. It is established that light in certain regions of the visible spectrum will be highly absorbed by specific targets, especially targets containing hemoglobin. Thus, as the concentration of hemoglobin in a target increases, so too does the absorption of these wavelengths. Whole blood has the highest concentration of hemoglobin of any tissue, making it the highest absorber of certain wavelengths of light. Secondly, it is also established that light is scattered by tissues and that this scattered light can be collected and analyzed.

As the needle enters the tissue, light will be transmitted into the skin. Backscattered light reflected by the tissue will be collected by the device and sent to a detector. When the needle enters a tissue that has low whole blood content, backscattering will be high and the detector will read a strong reflection signal. As the needle approaches a blood-containing vessel, the reflection signal will drop. If the needle enters or touches a blood vessel, the light will be mostly absorbed by the whole blood. In this case, the amount of backscattered light sent to the detector will be low, which will result in a sudden loss of signal.

Among other components, the system includes a light source and a detector. In certain embodiments, an LED source which emits light at a 568 nm wavelength was chosen as the light source because it allows differentiation between blood hemoglobin and muscle myoglobin, although in other embodiments an LED source which emits light with a wavelength in a range of 568-577 nm could be used. The LED source may be housed in the needle hub where it is coupled to a fiber optic probe by placing the end of the fiber adjacent to the LED source. Alternate embodiments of the fiber/LED connection include a rounded LED source or a ball lens attached to the end of the waveguide to increase the amount of light captured and delivered to the fiber. In still other embodiments, the LED source and/or the fiber may have other shapes.

As indicated above, the needle hub also houses an optical detector and the supporting electronics that capture and process the backscattered light. This detector is coupled to a similar fiber optic probe as the fiber attached to the light source and may use a similar coupling as those shown herein.

Needle or Cannula Gauge

In one embodiment, the device includes a 25 gauge needle with two fibers. One of the fibers is used to deliver light from the source to the tissue and the other fiber is used to collect light from the tissue and deliver it to the detector. The device may have interchangeable needles and cannulas, where the range of acceptable needle/cannular/tubular element gauges may be between 23 gauge and 30 gauge. The minimum inner diameter is 0.159 mm (the inner diameter of a 30 gauge needle) and the maximum outer diameter is 0.6414 mm (the outer diameter of a 23 gauge needle). The inner diameter needs to be sufficiently large to allow the material (e.g. dermal filler) to flow at a sufficient rate; certain materials including some types of dermal filler materials may have a high viscosity that requires a relatively large inner diameter to facilitate flow. The outer diameter in turn needs to be sufficiently small so that it does not injure the person receiving the injection. The balance between an inner diameter that is sufficiently large to accommodate flow of materials and an outer diameter that is sufficiently small to avoid tissue injury is further constrained by the need to provide space for the optical fibers and thus the outer diameter of the fibers is limited by these constraints.

In various embodiments, the placement of two 100 micron fibers in a 23-25 gauge needle/cannula (OD 0.6414 mm for 23 gauge to 0.5144 mm for 25 gauge) is not expected to impact filler delivery. The wall thickness of a standard needle is 0.15 mm (for a 23 gauge needle), 0.127 mm (25 gauge), and 0.1016 mm (27 gauge). There is a chance of decreasing the inner diameter of the 27 gauge needle (OD 0.4128 mm) when the fibers are included within the needle wall or in a groove formed in the wall but the impact should be minimal, and in certain embodiments the inner diameter may be reduced to 0.159 mm (i.e. comparable to that of a 30 gauge needle) from 0.210 mm (i.e. typical ID of a 27 gauge needle). At lower gauges (corresponding to larger needle cross-sectional sizes), there will be minimal impact of filler delivery when the inner diameter is reduced in order to accommodate the fibers. At higher gauges (corresponding to smaller needle cross-sectional sizes), such as 30 gauge (OD 0.3112 mm, ID 0.159 mm, wall 0.0762 mm), the use of two fibers will impact either or both of the outer diameter (OD) or the inner diameter (ID). Nevertheless, the disclosed device is less likely to be needed or used with smaller needles such as 30 gauge needles because such smaller needles are typically used on structures such as very fine lip lines and only at superficial levels close to the skin surface and away from the deeper blood vessels that have been associated with most adverse outcomes. In various embodiments, a 27 gauge needle and a 23-25 gauge cannula may be used with the system.

Wavelength Range

As noted above, in certain embodiments a preferred wavelength of light to transmit to the tissues is 568 nm, a wavelength which was chosen because it allows differentiation between blood hemoglobin and muscle myoglobin. Nevertheless, in various embodiments the range of usable wavelengths is between 400-600 nm, more preferably between 500-600 nm, and most preferably between 568-577 nm. Major hemoglobin absorption features are located in the 400-600 nm region because the major hemoglobin peaks are present in this wavelength region. Since longer wavelengths (e.g. longer than 600 nm) result in reduced scattering and given that there are fewer biological absorbers that will absorb the excitation light, the 500-600 nm range is more preferable than the 400-500 nm range. The most preferable region is between 568-577 nm where there is a significant difference in absorption between hemoglobin and myoglobin. Confinement of the light to a narrow spectral region is expected to increase the sensitivity of the device.

Fiber Diameter and Specification

In some embodiments, the diameter of the optical fibers may be 100 microns, although in other embodiments the fiber diameter may be smaller than 100 microns. In still other embodiments, larger diameter fibers (e.g. 200 microns) may also be used. In certain embodiments, the two fibers may be placed as close as possible to each other, whereas in other embodiments there may be space between the fibers. In addition, the numerical aperture (NA) of the fiber is generally in the range of 0.2-0.3, but in some embodiments can be as high as 0.5.

Both the NA of the fiber and the fiber spacing impact how far ahead of the needle the ends of the fiber can “see,” or detect. In general, with a smaller NA the fiber sees tissue that is further ahead of the end of the fiber, whereas with a larger NA the fiber sees tissue that is close to the end of the fiber. As a result, in some embodiments it may be possible for the system to provide a “warning” signal when the fiber ends and needle tip are close to, but not inside of, a structure such as a blood vessel as opposed to only being able to provide a “stop” signal after the needle tip has entered a blood-containing structure such as a blood vessel. That is, a particular combination of fiber spacing and fiber NA permit the detection point of the fibers to be set at a particular distance from the optical fibers, such that it is possible to detect tissue type (e.g. blood or muscle) at the particular distance from the needle tip, thereby allowing the detection system to “see” the tissue that is a certain distance away from the needle tip and in the path of the needle and issue a warning signal to the user before the needle enters a certain type of tissue.

In the current design, the use of two fibers allows optimization of fiber spacing, which in turn helps the device differentiate between muscle and blood. This is because of the path length that the light travels between the two fibers differs for muscle and blood. Therefore, fiber spacing impacts blood and muscle signal. In various experiments, it was found that placing the fibers next to each other gave better results, although a cladding around the fibers may have impacted true distance. Fiber spacing options, however, may be limited by device size.

The fibers can have an outer diameter (OD) of 125 μm (with a 105 μm core). In one particular embodiment, the fibers may be multimode fibers from Thorlabs (FG105UCA, or UM22-100 having a smaller coating diameter).

In certain embodiments, the needle or cannula can have 1, 2, 3, or other number of optical fibers associated therewith. As disclosed herein, factors such as the spacing between fibers and the numerical aperture (NA) of the fiber ends determine how far away from the ends of the fibers the tissue can be detected. In various embodiments, when the needle or cannula includes two optical fibers, when the fibers are spaced close to one another they do not detect the tissue until it is close to the fiber ends but the signal that is generated is higher. On the other hand, as the spacing between the fibers increases the fibers can detect tissue further away but the signal that is generated is lower. Accordingly, in some embodiments the needle or cannula may include three or more optical fibers with varied spacing therebetween. The fiber which emits light into the tissue may have a second fiber for detection that is closely spaced to provide high signal levels from nearby tissue and a third fiber for detection that is spaced further away to provide signals from tissues that are further away, albeit with lower signal levels; fourth or other fibers may provide signals with varying levels from varying distances from the needle or cannula.

The maximum spacing between any two fibers for a 25-gauge needle having an outer diameter of 0.474 mm is approximately 0.57 mm, assuming the use of 100 μm diameter fibers and assuming that the fibers are measured center to center. Thus, there is a limited range of fiber distances that can be implemented for small gauge needles. Note that as the fiber separation increases, deeper regions of the tissue are sampled, and the signal level drops considerably. On the other hand, maintaining contact between the illumination and collection fibers allows for maximum return signal to be collected at a shallow tissue depth.

A two-fiber arrangement significantly separates the internally reflected light from the tissue backscattered light. Adding a small amount of space between the fibers and/or a material that optically isolates the collection fiber from the delivery fiber, such as a coating (e.g. a metal or other coating) on the collection fiber, will result in a lower background signal.

For 30 gauge needles, for example, including optical fibers having large diameters (e.g. 100 μm) can be detrimental to fluid flow since the ID of the needle is so small. However, the use of tapered fibers can mitigate this issue where the light source is coupled into a large end of the fiber such that the fiber is tapered and the smaller end is still capable of transmitting light. The tapered portions of these fibers can be very small 10 μm or less.

It is possible to use a single fiber to transmit light from the source to the tissue and back from the tissue to the detector, although there are some challenges to this approach due to potential cross talk of light within a single fiber, whereas the use of two fibers limits cross talk. Nevertheless, in some embodiments a single fiber is used, preferably a multimode fiber with a small cladding and jacket thickness, to deliver and collect the back scattered light from the tissue, which allows the optical system to have a minimal cross-sectional footprint. In this embodiment, the forward traveling illumination light propagates along the waveguide until it reaches the tissue/fiber interface. At that point, a small fraction of the light may be reflected back through the fiber. The fiber may also include small scattering centers or defects in the fiber induced in either the drawing process or through handling that will also reflect light back through the fiber to the detector. The magnitude of these sources of backscattered or reflected light may be significantly higher than the magnitude of backscattered light from the tissue, which presents a further difficulty.

In one embodiment, to avoid cross-talk in the single fiber solution a circular polarizer or circular polarizing element may be placed at the tip of the fiber that would weight the detected light towards light that has scattered in the tissue, which would improve signal and facilitate the use of only one fiber.

In another embodiment which uses only a single fiber for light transmission and data collection, a dual clad fiber, also referred to as double clad fiber, may be used. In the dual clad fiber the inner core (˜6-10 μm) transmits the illumination light and the collected tissue light is collected by the inner cladding. Physical separation between the internal reflected light and collected tissue scattering may be achieved by use of physically blocking the image of the core projected on to the detector. The degree of cross talk between the core and inner cladding is a function of the ability to couple the illumination into the core only.

In still another embodiment which uses only a single fiber, light is shined directly on the face of the person who is undergoing injections without using the needle. In embodiments such as this, the light source may be directed toward the patient's face at a distance from the skin, or the light source may be held very close to, or even pressed against, the skin surface. If the end of the needle is located in a blood vessel, the blood will serve as a filter and will block the light that has entered through the skin on the face and scattered around in the tissue.

In yet another embodiment which uses only a single fiber, the inside of the needle may be coated with a film, such that the refractive index difference between the filler material being injected and the film coating will guide light in the film. This would facilitate using less of the lumen space with fibers or other optical transmission material.

Material Specification

In general, the disclosed device may be made using skin contact-compatible polymers that can be injection molded, such as polypropylene (PP) or Nylon 6-6, and materials that are opaque to prevent light leakage. Opacity and moldability are not necessary though, as the device can work with a translucent castable resin as well.

One particular embodiment of the device may include one or more of the following elements:
568 nm LED (500-650 nm): Lumileds LXZ1-PX01, power 100 mA (250 mA at 2.9 V);
Photodiode Detector (350-1100 nm): a Maxim Integrated MAX8610 Dev board together with a Vishay Semiconductor VEMD5080X01 photodiode;
Fibers: silica core with cladding and potential polyamide/acrylate coating such as Thorlabs FP200URT, 200 um core, NA 0.5 AND 100 um core, NA 0.2-0.3.
Detection filter with a range of 563-573 nm with peak at 568±5 nm (85% efficiency (Edmond Optics, 65-099).

Sterilization techniques may include exposure of the device to ethylene oxide gas (EtO) or alternatively autoclaving. However, gamma irradiation is not likely to work as it is incompatible with electronics. EtO is particularly suitable for use with materials such as Nylon or PP.

Signal Processing

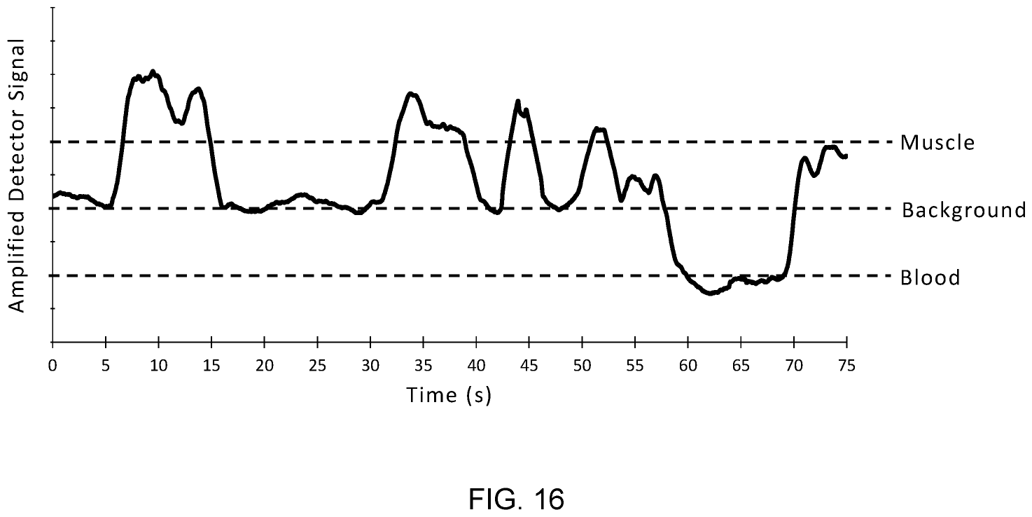

In certain embodiments, tissue identification may be based on the signal level (e.g. the level or intensity of reflected light returned from the tissue) achieving a particular threshold level. FIG. 16 shows results obtained from inserting a fiber-containing needle device into different biological tissues, where signal levels are recorded by the embodiment with source and detector waveguides are positioned adjacent to each other. The waveguides are 100-micron multimode fibers with a numerical aperture of 0.22. The light source was a broadband LED with a peak wavelength of 568 nm having a spectral width of greater than 50 nm. The optical signal was detected using a photodiode without spectral filtering or focusing elements. The data presented in FIG. 16 is an amplified analog signal from the detector as a function of time as the fiber-enabled needle encounters different tissue types and ambient background. From the data, clear distinction may be seen between levels of backscattered light received by the detector waveguide as it entered different tissue types. Signal processing algorithms can be applied to threshold this signal and send a second signal to alert a user when vascular penetration into blood is achieved. In some embodiments, signal processing routines may include a moving average to smooth the real-time data and to facilitate comparison of threshold levels or reference values. When the signal drops below the background signal, which indicates that the needle is in or near a blood vessel, the user will be alerted (e.g. using a visual and/or audible alert signal). Threshold or reference levels may be established using data from ex vivo tissue examination and further refined with in vivo experimental findings. Additional algorithms may be applied to establish and or refine the warning and stop signals. Threshold signaling can be further optimized to minimize false alarms.

Other Applications

While embodiments of the device using threshold signal levels are suitable for dermal filler applications, in other embodiments for other injection applications other signal processing procedures may be used to identify tissues and provide feedback to the user. For other applications, the tissue type may be detected based on return light based on the absorption and scattering coefficient. A ‘smart’ algorithm can inform the user of a likelihood of the presence of blood vessels based on data such as the absorption and scattering coefficient, which will be particularly suitable for needle guidance in addition to needle detection.

Disposability

In certain embodiments the device may be entirely disposable. However, in other embodiments the device may be designed such that certain parts would be disposable (e.g. the needle and related fluid-delivery components) while other parts would be separable and reusable, for example the device may include an external light source connected to the needle via fibers. In this particular embodiment, the needle and fibers would be disposable while the light source would be reusable. In embodiments such as these, the fibers may exit the needle hub and connect to an external “black box” which includes the light source and detector, where the fibers would plug into the box in a manner that would not require alignment. In still other embodiments the device may include an external power supply that is not disposable. In yet another embodiment, data may be transmitted from the device (for example wirelessly, e.g. via Bluetooth) and processed in an external environment (e.g. a smartphone app or other external computing system).

Alternative Configurations

Although many embodiments of the injection device disclosed herein provide an injection device in which a hub containing optical fiber(s), light source, and sensor(s) is detachably connected to a flange (which couples to a syringe barrel) which contains a power source, detection electronics, and an alert system, in other embodiments these components can be distributed between the hub and flange in different ways. Thus, in one embodiment the external case of the custom flange, which contains a power source, detection electronics, and alert system, may also contain components of the optical sensor assembly whereas the hub may include only the needle and optical fiber(s). In other embodiments, the hub and flange components including the power source, detection optics and electronics, and alert system may be integrated, along with the syringe barrel, to create a monolithic piece. In some embodiments, the syringe coupled to the injection system can be preloaded with an injection material (e.g. dermal filler) and packaged for immediate use. In another embodiment, due to the self-contained and modular nature of the injection system design, the customized flange may be designed as an ancillary component to be coupled onto a standard syringe of known geometry by medical personnel at the time of use, using a design which enables the custom flange to be secured to the syringe without removing the piston, which could compromise sterility of the injectable material.

Variations of Light Delivery

As noted briefly above, in some embodiments the needle and filler material may be used as the conduit through which light is transmitted to and/or from the tissue, which may be used in place of one or both of the parallel fibers. In embodiments such as these, a light delivery system may be attached to the needle hub or to the syringe itself. As described previously, in embodiments such as these the inside of the needle may be coated with a reflective material or other film such that the refractive index difference between the filler material being injected and the film coating will guide light in the film.

Also as noted above, in some embodiments the need for the light-transmitting fiber may be eliminated by shining light directly (without the use of a needle) onto the face of the subject who is receiving the injection. The detection fiber will collect the externally-transmitted light with an efficiency that will depend on the type of tissue in which the tip of the needle is located. For example, if the needle tip is located in a blood vessel, the blood will act like a filter and block the light that has entered through the facial skin and scattered around in the tissue.

In another embodiment the needle itself may be made of a light-transmitting material that is sufficiently rigid while still being capable of transmitting light. In embodiments such as these, the needle would not be steel, but rather a material such as pellethane or similar material that is firm enough to penetrate the skin, but has the right optical transmission properties. In particular embodiments the needle may include external cladding or coating.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. An injection apparatus, comprising:
a needle configured to be inserted into a tissue;
a light source to deliver light to the tissue to generate reflected light;
a detector to detect the reflected light from the tissue; and
a processor coupled to the detector and configured to:
analyze the reflected light from the tissue to identify a tissue type associated with the reflected light, and
provide an output to a user based on the identified tissue type.

2. The injection apparatus of claim 1, further comprising a signal mechanism including at least one of a light- or sound-generating mechanism, and
wherein the processor, when providing an output to a user based on the identified tissue type, is further configured to:
provide the output to the user using the signal mechanism based on the identified tissue type.

3. The injection apparatus of claim 1, further comprising a detector waveguide associated with the needle and optically coupled to the detector, wherein the detector waveguide is configured to transmit the reflected light from the tissue to the detector.

4. The injection apparatus of claim 3, further comprising a source waveguide associated with the needle and optically coupled to the light source, wherein the source waveguide is configured to transmit light from the light source to the tissue.

5. The injection apparatus of claim 3, wherein the needle comprises a longitudinal groove, and
wherein the detector waveguide is disposed within the longitudinal groove.

6. The injection apparatus of claim 3, wherein the needle comprises a longitudinal channel within the needle, and
wherein the detector waveguide is disposed within the longitudinal channel.

7. The injection apparatus of claim 4, wherein the needle comprises a longitudinal groove, and
wherein the source waveguide is disposed within the longitudinal groove.

8. The injection apparatus of claim 4, wherein the needle comprises a longitudinal channel within the needle, and
wherein the source waveguide is disposed within the longitudinal channel.

9. The injection apparatus of claim 4, wherein the detector waveguide is adjacent to the source waveguide.

10. The injection apparatus of any one of claims 3-9, wherein the detector waveguide comprises an optical fiber.

11. The injection apparatus of any one of claims 4-9, wherein the source waveguide comprises an optical fiber.

12. The injection apparatus of claim 4, wherein the detector waveguide is a first detector waveguide and wherein the detector is a first detector, the apparatus further comprising:
a second detector waveguide associated with the needle and optically coupled to a second detector,
wherein the second detector waveguide is configured to transmit the reflected light from the tissue to the second detector, and
wherein the second detector waveguide is closer to the source waveguide than the first detector waveguide.

13. The injection apparatus of claim 1, wherein the needle is coupled to a syringe barrel containing an injection material,
wherein the syringe barrel has a plunger disposed therein, and
wherein movement of the plunger into the barrel forces the injection material through a distal tip of the needle into the tissue.

14. The injection apparatus of claim 12, wherein the injection material comprises a dermal filler.

15. The injection apparatus of claim 1, wherein the light source has a wavelength between 568 nm and 577 nm.

16. The injection apparatus of claim 1, wherein the processor, when analyzing the reflected light from the tissue to identify a tissue type, is further configured to:
determine an intensity level of the reflected light from the tissue, and
identify the tissue type associated with the reflected light based on determining the intensity level of the reflected light from the tissue.

17. The injection apparatus of claim 16, wherein the processor, when determining an intensity level of the reflected light from the tissue, is further configured to:
compare the intensity level of the reflected light to a reference value, and
identify the tissue type associated with the reflected light based on comparing the intensity level of the reflected light to the reference value.

18. The injection apparatus of claim 1, wherein the needle is between 23 gauge and 30 gauge.

19. The injection apparatus of claim 1, wherein the needle is disposed within a cannula.

20. The injection apparatus of claim 1, wherein the apparatus is a standalone device.

21. The injection apparatus of claim 1, further comprising a hub and a flange,
wherein the needle, the light source, and the detector are associated with the hub, and
wherein the processor is associated with the flange.

22. The injection apparatus of claim 21, wherein the hub is detachably connected to the flange.

23. The injection apparatus of claim 1, further comprising a hub and a flange,
wherein the needle is associated with the hub, and
wherein the light source, the detector, and the processor are associated with the flange.

24. The injection apparatus of claim 23, wherein the hub is detachably connected to the flange.

25. The injection apparatus of claim 1, wherein the needle is coupled to a syringe barrel with a plunger disposed therein,
wherein the plunger is withdrawn to draw fluid from the tissue through the needle.

26. A hub for use with an injection apparatus, comprising:
a needle configured to be inserted into a tissue;
at least one waveguide associated with the needle; and
a connector for detachably connecting the hub to a flange.

27. The hub of claim 26, further comprising a light source and a detector,
wherein the light source is configured to deliver light to the tissue to generate reflected light, and
wherein the detector is configured to detect the reflected light from the tissue.

28. The hub of claim 27, further comprising a source waveguide and a detector waveguide,
wherein the detector waveguide is associated with the needle and optically coupled to the detector and is configured to transmit the reflected light from the tissue to the detector, and
wherein the source waveguide is associated with the needle and optically coupled to the light source and is configured to transmit light from the light source to the tissue.

29. A method for injection, comprising:
providing an injection apparatus comprising:
a needle configured to be inserted into a tissue,
a light source to deliver light to the tissue to generate reflected light, and
a detector to detect the reflected light from the tissue;
analyzing, using a processor coupled to the detector, the reflected light from the tissue to identify a tissue type associated with the reflected light; and
providing, using the processor, an output to a user based on the identified tissue type.

30. The method of claim 29, wherein providing an output to a user based on the identified tissue type further comprises:
providing the output to the user using a signal mechanism based on the identified tissue type,
the signal mechanism including at least one of a light or sound generating mechanism.

31. The method of claim 29, further comprising transmitting the reflected light from the tissue to the detector using a detector waveguide,
wherein the detector waveguide is associated with the needle and optically coupled to the detector.

32. The method of claim 31, further comprising transmitting light from the light source to the tissue using a source waveguide,
wherein the source waveguide is associated with the needle and optically coupled to the light source.

33. The method of claim 31, wherein the needle comprises a longitudinal groove, and
wherein the detector waveguide is disposed within the longitudinal groove.

34. The method of claim 31, wherein the needle comprises a longitudinal channel within the needle, and
wherein the detector waveguide is disposed within the longitudinal channel.

35. The method of claim 32, wherein the needle comprises a longitudinal groove, and
wherein the source waveguide is disposed within the longitudinal groove.

36. The method of claim 32, wherein the needle comprises a longitudinal channel within the needle, and
wherein the source optical fiber is disposed within the longitudinal channel.

37. The method of claim 32, wherein the detector optical fiber is adjacent to the source optical fiber.

38. The method of any one of claims 31-37, wherein the detector waveguide comprises an optical fiber.

39. The method of any one of claims 32-37, wherein the source waveguide comprises an optical fiber.

40. The method of claim 32, wherein the detector waveguide is a first detector waveguide and wherein the detector is a first detector, the method further comprising:
transmit the reflected light from the tissue to a second detector,
wherein a second detector waveguide is associated with the needle and optically coupled to the second detector, and
wherein the second detector waveguide is closer to the source waveguide than the first detector waveguide.

41. The method of claim 29, wherein the needle is coupled to a syringe barrel containing an injection material, and
wherein the syringe barrel has a plunger disposed therein, and
wherein the method further comprises:
moving the plunger into the barrel to force the injection material through a distal tip of the needle into the tissue.

42. The method of claim 41, wherein the injection material comprises a dermal filler.

43. The method of claim 29, wherein the light source generates wavelengths of light between 568 nm and 577 nm.

44. The method of claim 29, wherein analyzing the reflected light from the tissue to identify a tissue type further comprises:
determining an intensity level of the reflected light from the tissue, and
identifying the tissue type associated with the reflected light based on determining the intensity level of the reflected light from the tissue.

45. The method of claim 44, wherein determining an intensity level of the reflected light from the tissue further comprises:
comparing the intensity level of the reflected light to a reference value, and
identifying the tissue type associated with the reflected light based on comparing the intensity level of the reflected light to the reference value.

46. The method of claim 29, wherein the needle is between 23 gauge and 30 gauge.

47. The method of claim 29, wherein the needle is coupled to a syringe barrel with a plunger disposed therein, the method further comprising:
withdrawing the plunger to draw fluid from the tissue through the needle.

48. A dermal filler apparatus, comprising:
a barrel having a proximal end and a distal end;
a plunger sliding within a portion of the body;
a plunger handle mounted to the plunger;
a needle extending from the distal end of the barrel;
a light source extending from the distal end of the barrel;
an optical detector extending from the distal end of the barrel;
an indicator; and
a processor.

49. The dermal filler apparatus of claim 48, wherein the indicator comprises a signal light.

50. The dermal filler apparatus of claim 48, wherein the indicator comprises an audio signal.

51. The dermal filler apparatus of claim 48, wherein the indicator comprises a vibrator.

52. The dermal filler apparatus of claim 48, wherein the needle is interchangeably coupled to the insertion apparatus.

53. The dermal filler apparatus of claim 48, wherein the optical detector comprises a photodiode detector.

54. The dermal filler apparatus of claim 48, wherein the light source comprises a 568 nm light emitting diode (LED).

55. The dermal filler apparatus of claim 48, wherein the optical detector comprises a 100 micron fiber.

56. The dermal filler apparatus of claim 48, wherein the needle comprises:
a needle wall defining a conduit;
a lighting wire;
detector wire.

57. The dermal filler apparatus of claim 56, wherein the lighting wire and the detector wire are embedded in the needle wall.

58. The dermal filler apparatus of claim 56, wherein the lighting wire and the detector wire are external to the needle wall.
